(19) United States
(12) Patent Application Publication
(10) Pub. No.: US 2014/0163397 A1
(43) Pub. Date: Jun. 12, 2014

(54) Gas Exchange Testing and Auxiliary Gas Delivery Apparatus

(71) Applicant: Shape Medical Systems, LLC, St. Paul, MN (US)

(72) Inventors: Stephen T. Anderson, North Oaks, MN (US); Dean J. MacCarter, Englewood, CO (US); Bruce D. Johnson, Rochester, MN (US); Tyler J. Bahr, Hudson, WI (US)

(73) Assignee: Shape Medical Systems, LLC, St. Paul, MN (US)

(21) Appl. No.: 14/099,595

(22) Filed: Dec. 6, 2013

Related U.S. Application Data
(60) Provisional application No. 61/734,463, filed on Dec. 7, 2012.

(51) Int. Cl. A61B 5/0205

(52) U.S. Cl. CPC A61B 5/0205; A61B 5/0833
USPC 600/484; 600/531

(57) ABSTRACT

A gas exchange testing system has an apparatus for delivery of an auxiliary gas during a gas exchange test. Measurements representing physiological parameters of a patient are made during the gas exchange test. The auxiliary gas is delivered so that a gas exchange test can be performed on a patient who requires auxiliary gas. Additionally, the auxiliary gas is delivered so that gas exchange testing can determine if the auxiliary gas is beneficial and which concentration of the auxiliary gas is most beneficial. A testing process compares measurements under a condition where the auxiliary gas is delivered and under a condition where it is not delivered. Another testing process compares measurements under a condition where one auxiliary gas is delivered and under a condition where a different auxiliary gas is delivered.

### RELATED APPLICATION

This application claims the benefit of U.S. Patent Application Ser. No. 61/734,463 filed on Dec. 7, 2012, the entirety of which is hereby incorporated by reference.

### BACKGROUND

Gas exchanging testing is useful for assessing the characteristics of a patient's cardiac and pulmonary systems. During gas exchange testing, the patient breathes through an interface that is used to determine the volume of breath inspired and expired. Additionally, the interface may also be used to determine the concentrations of various gases, such as oxygen (O₂) and carbon dioxide (CO₂), in the expired breath. Various properties of the patient's cardiac and pulmonary systems can be determined from these measurements.

Cardiac catheterization is the most important diagnostic test for pulmonary arterial hypertension (PAH) patients. It gives an accurate assessment of pulmonary artery pressures and cardiac output from which the pulmonary vascular resistance and transpulmonary pressure gradient can be calculated. And with mild exercise, cardiac catheterization can help to detect and define intracardiac shunts, rule out the presence of left heart disease, and help guide therapy.

Vasoreactivity testing can be combined with right heart catheterization to ascertain whether the PAH is “vasoreactive” or “fixed”. This finding is critical to both making transplant decisions and determining prognosis. Inhaled nitric oxide, intravenous adenosine, or prostacyclin are frequently used to determine vasoreactivity. When the mean pulmonary artery pressure decreases by at least 10 to 40 mmHg or less with an increase or unchanged cardiac output with acute vasodilator challenge, the patient is labeled “vasoreactive” or a “responder”. A positive vasoreactivity test suggests a response to drug therapy and good prognosis.

Pulmonary hypertension (PH) in heart failure (HF) patients is “postcapillary” characterized by an elevated pulmonary capillary wedge pressure (PCWP) and pulmonary vascular resistance (PVR). Initially, PH in HF is “vasoreactive” and is readily reversed acutely with vasodilator challenge. Over time, PH becomes “nonvasoreactive” or “fixed” with reduced responsiveness to pharmacologic treatments. For this reason, such patients need to be monitored closely earlier so treatment changes can be effectively initiated. This will become increasingly important when the Affordable Healthcare Act is implemented, forcing healthcare provider organizations to keep people out of the hospital as incentives shift from volume to quality.

The presence of pulmonary hypertension carries with it a poor prognosis, irrespective of the causes. When PH complicates left heart failure, both morbidity and mortality are increased. Patients complain of worsening fatigue and dyspnea and declining exercise tolerance. The peak exercise oxygen consumption correlates inversely with mean pulmonary pressure and pulmonary vascular resistance and correlates directly with resting right ventricular ejection fraction. Recently, retrospective analysis of databases for PAH and HF patients containing simultaneously collected cardiopulmonary exercise variables and cardiac catheterization lab measurements and cardiac ultrasound measurements suggest strong correlations with Cardiac Index and Right Ventricular Systolic Pressure measurements (Kim, Anderson, MacCarter, Johnson, A Multivariable Index (MVI) for Grading Exercise Gas Exchange Severity in Patients with Pulmonary Arterial Hypertension and Heart Failure).

The main disadvantage of collecting cardiac catheterization measurements is patient discomfort, high cost, and risk of complications from the procedure. There are also methodological problems of the procedure itself: 1) patients are supine, often under mild sedation, and under emotional stress, which by itself can influence hemodynamics (blood pressure, heart rate, and cardiac output); the response to these conditions can be quite variable across patients making reproducible measures on subsequent days quite difficult, 2) contraction of leg muscles or movement in the legs (e.g., bending), shifting of blood volume centrally may also influence the measurement, 3) large respiratory variations in the pressure signal, thus the timing and technique of making a measurement is critical, and 4) an assumption exists that PCWP is always reflective of left atrial pressure and thus the mean Pulmonary Arterial Pressure (PAP)—PCWP is a good reflection of pressure change across the pulmonary vasculature; in reality the measure of PVR only includes larger arteries and arterioles and ignores the largest part of the pulmonary circulation (capillaries and veins) which has been shown to have contractile potential and could play a role in dictating PVR.

Another problem with right heart catheterization measurements is that the sensitivity using simple Fick principle measurements based on mean PAP and PCWP to determine Cardiac Output (CO) does not show the physician much in the way of a dynamic vascular response since the measurements can only be made every 1 to 2 minutes at best, thus blunting the actual “real time” dynamic hemodynamic patterns that may occur. Consequently, the physician is looking for a change that has been blunted or not properly depicted by too infrequent of a measurement sequence that describes inaccurately the true vasoreactive response.

### SUMMARY

The present disclosure, to a large extent, obviates the problems discussed in the foregoing for each of the phases described above. The physiology supportive of the present disclosure involves the relationship of the pulmonary circulation and gas exchange in the lungs that will readily reflect upon ventricular filling pressures, pulmonary venous flow, and ventilation to perfusion matching in the lungs (see also Definitions). A sound physiologic basis exists to support the theory that the oxygen pulse (O₂ Pulse), end-tidal partial pressure of CO₂ (PetCO₂), gas exchange capacitance (GxcAp), ventilatory equivalents of CO₂ (VE/VCO₂), and inspiratory drive (VT/tᵢ) are key parameters to assess pump function of the heart and the efficiency of gas exchange in the lungs. Any therapy, which reduces stroke output of the heart, may cause a volume load or increased preload on the heart, thus affecting the pulmonary venous blood flow gradient and ventilation to perfusion matching in the lungs. When ventilation to perfusion is mismatched, the PetCO₂, O₂ Pulse, and Gx_{CAP} will be reduced and VE/VCO₂ and VT/tᵢ will be increased. Because gas exchange measurements are made on a “breath-by-breath” basis, physiologic changes resulting from vasoreactivity testing are observable more or less instantaneously, thus they can be used to guide the decision making process in either case.

As has been aforementioned, vasodilators, including inhaled nitric oxide (NO), intravenous adenosine, or prostacyclin, are frequently used to determine vasoreactivity. For the purpose of describing the present disclosure, inhaled NO will typically be discussed, although it should be understood that any other appropriate inhaled or intravenous agents including intravenous adenosine or prostacyclin could be substituted without altering the intent of the methods described. It should also be noted that the present disclosure can be utilized to test a patient while recumbent in the catheterization lab, recumbent outside the catheterization lab, or during upright exercise outside of the catheterization lab.

In general terms, this disclosure is directed to the field of medical diagnosis and therapy monitoring and, more specifically, to a system for evaluating patients using gas exchange testing. In one possible configuration and by non-limiting example, the gas exchange testing system is configured to include an auxiliary gas delivery apparatus. Various aspects are described in this disclosure, which include, but are not limited to, the following aspects.

One aspect is a system for delivering an auxiliary gas to a patient during a gas exchange test, comprising: a pneumotach; an analyzer configured to determine a rate of a gas flow through the pneumotach; and a directional valve having an inspiratory port and an expiratory port, the directional valve configured to connect the inspiratory port to the pneumotach during patient inspiration and to connect the expiratory port to the pneumotach during patient expiration.

Another aspect is a method of performing a gas-exchange test on a patient, comprising: providing an auxiliary gas to the patient; and evaluating, using a device, a plurality of breaths of the patient while the patient is receiving the auxiliary gas, wherein evaluating a breath of the plurality of breaths comprises: determining a flow of gas expired by the patient during the breath; and determining at least a portion of a composition of the gas expired by the patient during the breath.

Yet another aspect is a method of performing a gas-exchange test on a patient, comprising: providing supplemental oxygen to the patient; evaluating, using a device, a plurality of breaths from a rest phase and an exercise phase, the rest phase comprising a plurality of breaths while the patient is resting, the exercise phase comprising a plurality of breaths while the patient is exercising, wherein evaluating a breath comprises: determining a concentration of oxygen in the gas inspired by the patient during the breath; determining a flow of gas expired by the patient during the breath; determining a concentration of oxygen in the gas expired by the patient during the breath; and determining a concentration of carbon dioxide in the gas expired by the patient during the breath; and calculating at least one of a ventilatory efficiency slope value and an oxygen uptake efficiency slope value for the patient based on the breaths evaluated during the rest phase and the breaths evaluated during the exercise phase.

### BRIEF DESCRIPTION OF THE DRAWINGS

FIG. 1 is a schematic drawing that illustrates an embodiment of a gas exchange testing system.

FIG. 2 is a drawing of a breathing circuit and cart of an embodiment of the gas exchange testing system of FIG. 1.

FIG. 3 is a schematic drawing illustrating the functional components of an embodiment of the breathing circuit of FIG. 2.

FIG. 4 is a schematic drawing that illustrates an alternative embodiment of a breathing circuit for the gas exchange testing system of FIG. 1.

FIG. 5 illustrates an example process of evaluating the effect of an auxiliary gas using the breathing circuit of FIG. 3.

FIG. 6 illustrates an example process for evaluating the effect of various different concentrations of supplemental O₂ using the breathing circuit of FIG. 4.

FIG. 7 illustrates an example data table of data acquired during the example process of FIG. 5.

FIG. 8 illustrates another example of data table and chart of data acquired during the example process of FIG. 5.

FIG. 9 illustrates an example report that includes some of the data collected using the testing protocol of FIG. 6.

FIG. 10 illustrates an example process of estimating FiO₂ received by a patient breathing supplemental oxygen outside of a laboratory environment is illustrated.

FIG. 11 illustrates two example relationships between F_{b} and VT used in the process of FIG. 10.

### DETAILED DESCRIPTION

This disclosure relates generally to the field of medical diagnosis and therapy monitoring and, more specifically, to a method for non-invasively determining whether pulmonary arterial hypertension patients or heart failure patients with associated pulmonary hypertension are “vasoreactive” or “fixed.” The same method and related breathing apparatus can be used to evaluate the effectiveness of breathing a mixture of room air and oxygen for supplemental oxygen therapy. The disclosed method enables physicians to more rapidly and more cost effectively make treatment decisions and determine prognoses. In addition, the present disclosure provides feedback during long-term follow-up in PAH patients and HF patients with associated PH who are being treated with a variety of available pharmaceutical vasodilators and chronic obstructive pulmonary disease (COPD) patients who are being treated with supplemental oxygen.

The following contains definitions and explanations of certain terms as used in the present context.

Carbon Dioxide Production (VCO2)—The volume of CO₂ expelled in expired air. VCO₂ is often calculated as a rate (e.g., ml/minute).

End-Tidal Partial Pressure of CO₂ (PetCO₂)—The partial pressure of carbon dioxide at the end of expiration, or the highest value of the partial pressure of CO₂ (PCO₂) during a single expiration.

Fraction of inspired oxygen concentration (FiO₂)—the fraction or percentage of oxygen in the space being measured. The FiO₂ is used to represent the percentage of oxygen participating in gas-exchange.

Gas Exchange capacitance (Gx_{CAP})—Invasive measures of PV capacitance (e.g., stroke volume/pulmonary arterial pressure, mPpa=Pv_{CAP}) are predictive of survival in PH, Previous studies have suggested that PetCO₂ in particular seems to track the changes in pulmonary vascular pressure with exercise, thus PetCO₂ can be used as a non-invasive metric of mPpa. To allow the correct directional change, 1/PetCO₂ is used in the calculation of Gx_{CAP}. In addition, O₂ Pulse tracks the stroke volume response to exercise in HF and thus can be used as a non-invasive estimate of stroke volume for calculating the Gx_{CAP}=O₂ Pulse*PetCO₂. Woods, 2012.

Inspiratory Drive (VT/tᵢ)—Tidal volume (VT) is the volume of an average breath; inspiratory time (tᵢ) is the average time it takes to inspire. The ratio has been used as an index of ventilatory drive (the combined stimulation to breathe).

Minute Ventilation (VE)—The minute ventilation is the volume of gas inspired during a minute.

Oxygen Pulse (O₂ Pulse)—O₂ pulse is calculated by dividing VO₂ (ml/min) by heart rate and is useful as an indirect measure of combined cardiopulmonary oxygen transport. O₂ pulse is correlated to the product of stroke volume and arteriovenous O₂ difference. The circulatory adjustments that occur during exercise (e.g., widening arteriovenous O₂ difference, increased cardiac output, and redistribution of blood flow to the working muscle) increase the O₂ Pulse. Conversely, O₂ Pulse is reduced by conditions that reduce stroke volume. Fitter patients have a higher maximal O₂ pulse. Patients with heart disease have a lower maximal O₂ Pulse. Generally, O₂ pulse is higher in a healthy or fit patient compared to a less healthy or less fit patient under the same workload. V. Froelicher, J. Myers, et al., Exercise and the Heart. Mosby-Year Book, Inc. 1993, p. 38.

Oxygen Uptake Efficiency Slope—The slope of the line of linear regression obtained from a plot of VO₂ against log VE.

Retrograde Pump Function—Filling of the heart occurs during the relaxation part of the cardiac cycle and the atrial contraction. Filling pressure and the volume of blood that returns to the heart during diastole are termed preload. Any forward pump failure of the heart can increase the preload or retrograde flow into the atrium of the heart to undesirable levels which, in turn, has an adverse retrograde effect on the pulmonary pressure dependent flow gradient and hence, gas exchange in the lung.

Ventilation-Perfusion Coupling (PECO₂/PETCO₂)—Ventilation-perfusion coupling is a ratio that is calculated by dividing the mixed expired pressure of carbon dioxide (PECO₂) by the end-tidal partial pressure of CO₂ (PETCO₂). Gas exchange is most efficient when there is a precise coupling between ventilation (the amount of gas reaching the alveoli) and perfusion (the blood flow in pulmonary capillaries). The ratio of ventilation-perfusion coupling quantifies this coupling. Generally, the partial pressure of CO₂ in the alveoli controls the diameter of the bronchioles. As the CO₂ increases in some areas, the passageways servicing those areas dilate to allow more CO₂ to be eliminated.

Conversely, in areas with less CO₂, the passageways restrict. The partial pressure of O₂ causes similar responses. Alveolar ventilation and pulmonary perfusion, are always synchronized, as a result of the modifications. When there is poor alveolar ventilation, there are low oxygen and high carbon dioxide levels in the alveoli. This causes the pulmonary capillaries to constrict and the airways dilate, which better couples the airflow and blood flow. Alternatively, when the partial pressure of O₂ is high and the partial pressure of CO2 is low, the respiratory passageways constrict and there is a flushing of blood into the pulmonary capillaries. These homeostatic mechanisms provide the most appropriate ventilation-perfusion coupling for efficient gas exchange. E. Marieb, Human Anatomy and Physiology. Benjamin/Cummings Publishing Company, 1992, p. 749.

Ventilatory Efficiency Slope—The slope of the line of linear regression obtained from a plot of VE against VCO₂.

Ventilatory Equivalent for carbon dioxide (VE/VCO₂)—The VE/VCO₂ is calculated by dividing ventilation (L/min) by VCO₂ (L/min). VE/VCO₂ is a ratio that represents the amount of ventilation required to expire a certain level of CO₂ produced by the patient's metabolizing tissues. Metabolic CO₂ stimulates ventilation during exercise. Accordingly, VE and VCO₂ generally track one another. After an initial drop in during exercise, VE/VCO₂ typically does not significantly increase significantly throughout the first phase of sub-maximal exercise until the ventilator threshold has been reached. But in a patient with chronic heart failure, VE/VCO₂ is comparatively higher than in healthy patients. High VE/VCO₂ values are characteristic of the abnormal ventilatory response to exercise in heart failure patients. Ibid Froelicher.

Ventilatory Oxygen Uptake (VO₂)—The volume of O₂ extracted from inspired air. VO₂ is often calculated as a rate (e.g., ml/minute).

Various embodiments will be described in detail with reference to the drawings. Reference to various embodiments does not limit the scope of the claims attached hereto. Additionally, any examples set forth in this specification are not intended to be limiting and merely set forth some of the many possible embodiments for the appended claims.

Physiologic parameters of a patient and changes to those parameters are measured using a gas exchange testing system. The gas exchange testing system measures selected variables associated with oxygen consumption, carbon dioxide production, ventilation, and heart rate. A cardiopulmonary exercise testing system is an example of a gas exchange testing system.

During the acute phase of evaluation, the dependent variables, PetCO₂, O₂ Pulse, VE/VCO₂, VT/tᵢ, Gx_{CAP}, and HR are measured during steady-state conditions at rest and during exercise. Upon completing the exercise test, the additional variables comprised of Ventilatory Efficiency and Oxygen Uptake Efficiency slopes and the V/Q ratio can also be computed. The independent variables are 1) room air breathing, 2) breathing a mixture of room air with a concentration of NO (e.g., 40 ppm), and 3) supplemental oxygen breathing (e.g., 29.3-100%). Changes made by the physician to an independent variable have the effect of changing the ventricular filling and stroke output of the heart and, in turn, altering the ventilation-perfusion coupling or the V/Q ratio. As the local autoregulatory mechanisms seek to restore the synchronization of alveolar and pulmonary perfusion, the dependent variables rapidly change. These changes to the dependent variables are measured. In some embodiments, the measured values for the dependent variables are automatically scaled and displayed to provide visual feedback to the physician during periods of room air breathing and during NO or supplemental oxygen breathing. In doing so, the physician is provided with a true, physiologic assessment of the patient's condition resulting from changes made to an independent variable at any point in time during the procedure.

By also providing a chronic assessment of the aforementioned independent variables over time, the physician can better understand the consequence of any given pharmaceutical therapeutic action. By providing a closed-loop system of action (therapy) and physiologic response (to therapy), the quality of prescribing vasodilator pharmaceuticals will be increased and the cost reduced.

The data gathering aspect of the disclosure involves known techniques and analyses and it is the aspects of processing, combining, and presenting the data in which the disclosure enables an observer to gain new and valuable insight into the present condition and therapy trends in patents. Thus, in accordance with the preferred method, a dynamic cardiopulmonary analysis is displayed for each data set. The performance of such a test is well understood by individuals skilled in the art, and no further explanation of this.

Equipment

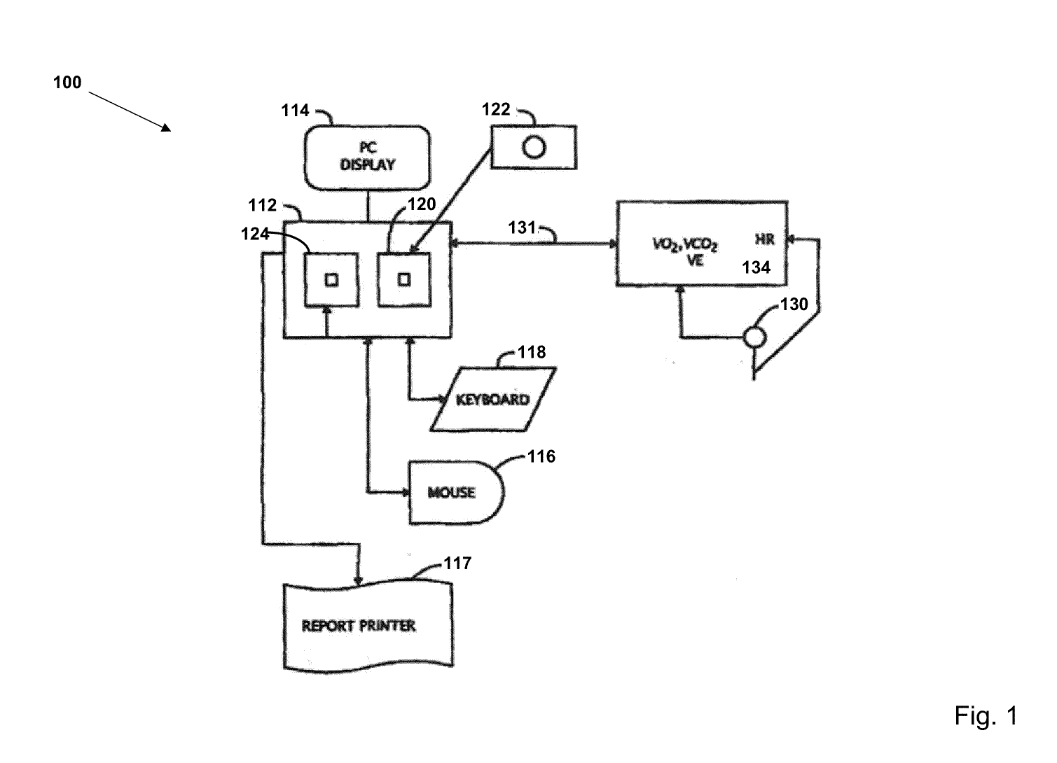

Referring now to FIG. 1, an embodiment of a gas exchange testing system 100 is shown. The gas exchange testing system 100 includes a computing device 112 and a gas exchange measurement device 134. Also shown is a patient or subject 130.

In some embodiments, the computing device 112 includes a display terminal 114 with an associated mouse 116, report printer 117, and keyboard 118. The system further includes a storage handler 120 with an associated memory storage device 122. As is well known in the art, the storage handler 120 input/output interfaces comprise read/write devices for reading, deleting, adding, or changing information stored on a machine-readable medium, e.g., a thumb drive, and for providing signals which can be considered as data or operands to be manipulated in accordance with a software program loaded into the RAM or ROM memory (not shown) included in the computing device 112. In some embodiments, the memory storage device 122 includes non-transitory storage devices. The computing device also includes a processor 124.

In some embodiments, the gas exchange test is a cardiopulmonary exercise test. In these embodiments, the exercise protocol for either the acute assessment or the chronic assessment includes exercise equipment (not shown), such as a bicycle ergometer, stair step, or treadmill. The subject 130 uses the exercise equipment during a portion of the test. During the gas exchange test, the gas exchange measurement device 134 measures various physiological parameters associated with the subject. In some embodiments, these physiological parameters include heart rate (HR), respiratory rate (RR), ventilation (VE), rate of oxygen uptake or consumption (VO₂), carbon dioxide production (VCO₂), and oxygen saturation (SpO₂). In other embodiments, other physiological parameters are measured as well. The gas exchange measurement device 134 is an example of an analyzer.

In some embodiments, the physiological data that is measured by the gas exchange measurement device 134 is transmitted to the computing device 112 via a conductor 131, such as a cable. In other embodiments, the physiological data is transmitted to the computing device wirelessly. In other embodiments, other communication devices are used to transmit the physiological data to the computing device 112. In some embodiments, the display terminal 114 displays the physiological data or other values derived from the physiological data.

The computing device 112 may comprise a personal computer, a dedicated microcontroller configured to acquire the measurements and process those measurements, a mobile computing device, such as a smart phone or tablet, or a server computer. Therefore, the further detailed description will be made independent of the type and characteristics of the computing device 112.

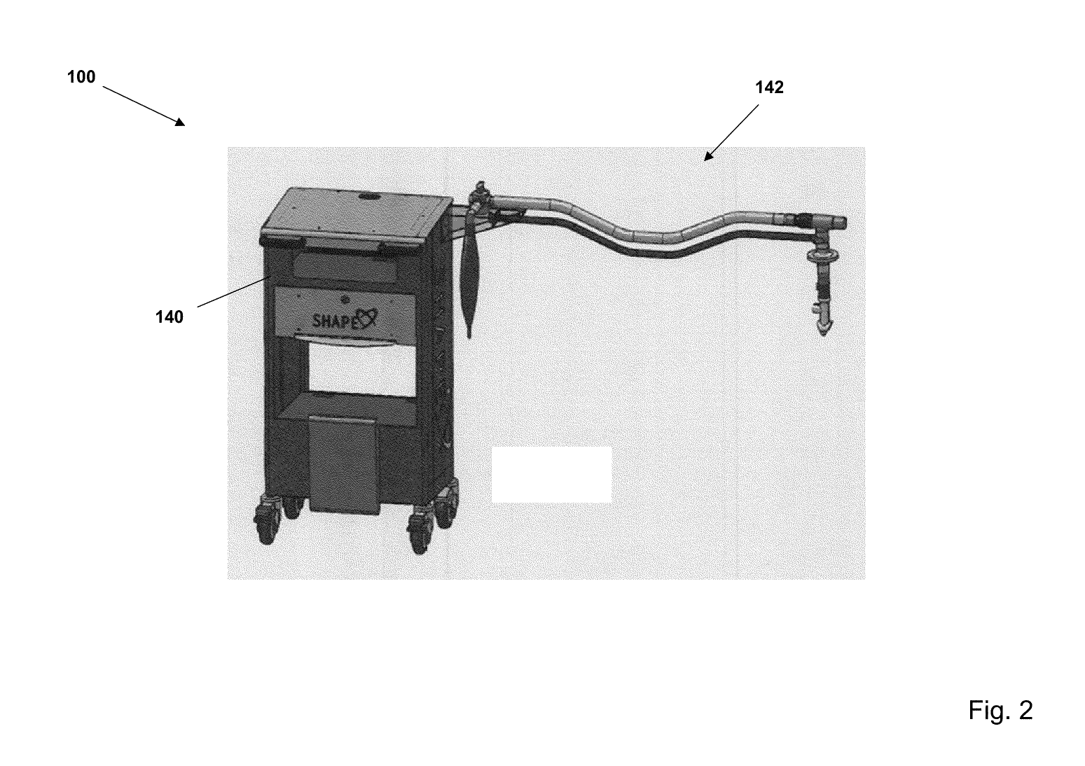

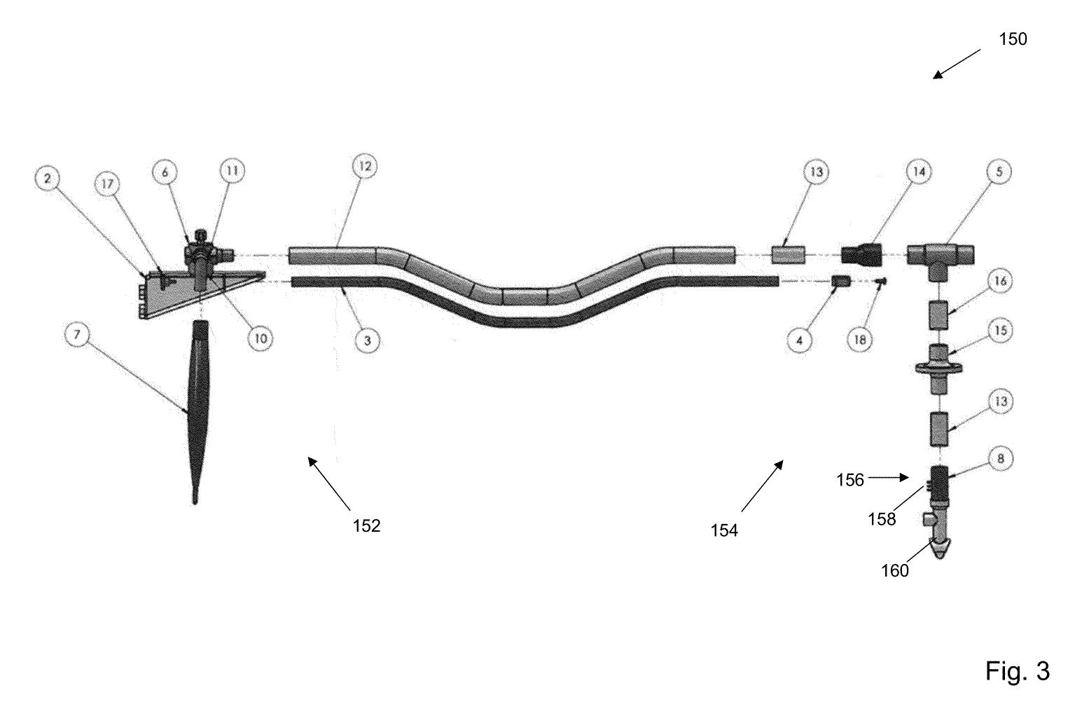

Referring now to FIG. 2, additional elements of an embodiment of the gas exchange testing system 100 are shown. Here, the gas exchange testing system 100 includes a cart 140 and a breathing circuit 142. In some embodiments, the breathing circuit 142 is configured to switch the source of gas for inspiration. In some embodiments, the breathing circuit 142 is attached to the cart 140. The breathing circuit 142 is illustrated and described in greater detail with respect to FIG. 3.

Referring now to FIG. 3, a close-up schematic view of the embodiment of the breathing circuit 142 is illustrated. The breathing circuit 142 is configured to switch the source of gas for inspiration from room air to room air with a selectable concentration of an auxiliary gas (e.g., NO or supplemental oxygen). The breathing circuit is described with respect to a cart side 152 and a patient side 154.

At the cart side 152 of the circuit 142: A cart bracket 2 is attached to the cart. A three-way stopcock 6 is mounted on the bracket 2. The stopcock 6 is a three-way valve and includes two selectable ports and one common port. The stopcock 6 is configured to connect the common port to one or the other of the selectable ports. The stopcock 6 includes a valve indicator switch to select one or the other of the selectable ports. Flow is permitted between the common port and the selected port, and flow is occluded for the non-selected port.

Attached to one of the selectable ports of the stopcock 6 is a breathing bag 7. In some embodiments, the breathing bag 7 is connected to the stopcock 6 via an elbow connector 10. The breathing bag 7 is connected to an auxiliary gas source and blender configured to deliver the desired mixture of room air and the auxiliary gas. An auxiliary gas is any gas other than room air and includes mixtures of room air with another gas. In some embodiments, the auxiliary gas is NO. In other embodiments, the auxiliary gas is supplemental oxygen. The other selectable port of the stopcock 6 is open to room air.

The common port of the stopcock 6 is connected, via a coupler 11, to a breathing tube 12 that connects to the remaining components of the circuit. In some embodiments, the breathing tube 12 has a twenty-two millimeter inner diameter. In other embodiments, the breathing tube 12 has a smaller or larger inner diameter. A fastener 17 is mounted on the cart bracket 2, which is used to connect one end of a gooseneck 3 which provides a flexible, mechanical support means for the remaining components of the circuit (e.g., the components on the patient side 154). In some embodiments, the fastener 17 is a thumbscrew and is mounted on the underside of the cart bracket 2.

At the patient side 154 of the circuit 142: On the other end of the breathing tube 12, a coupler 13 and reducer 14 are connected in series with a two-way directional valve 5. The opposite side of the valve is open to room air and is used to vent the patient's expired air. The bottom port is connected to a coupler 16, which is connected to a bacterial filter 15, which is connected to another coupler 13, which is connected to the expiration side of a patient interface 8. The patient interface 8 includes a fixed orifice differential pressure pneumotach 156, sample port 158, and patient mouthpiece 160. The pneumotach 156 and sample port 158 of the patient interface 8 are connected to the gas exchange measurement device 134 to facilitate determining gas flow rate and composition (for example, the concentration of O₂ or CO₂). A gripper clip 4 is attached to the other end of the gooseneck 3, which is connected to the reducer 14, which supports the weight of the two-way valve 5, bacterial filter 15, patient interface 8, and couplers 13, 16. In addition to providing support, the gooseneck 3 can be bent to accommodate a comfortable positioning of the circuit for recumbent patient or an exercising patient breathing on the mouthpiece. Some embodiments do not include all of the parts described above. For example, in some embodiments a mask is included rather than the mouthpiece 160.

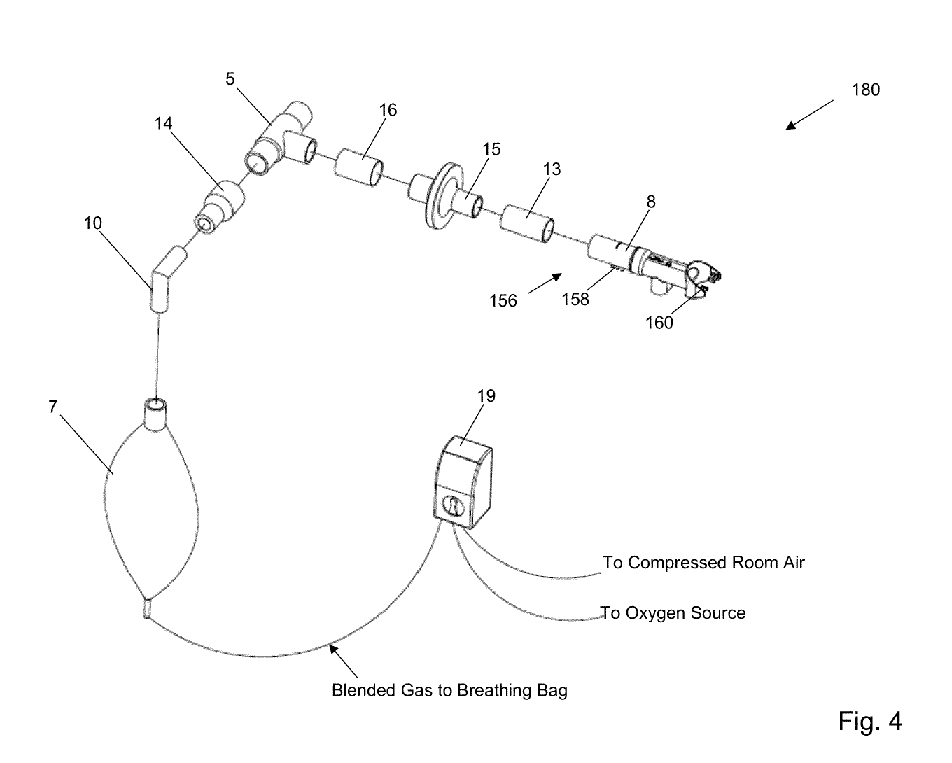

Referring now to FIG. 4, a simplified breathing circuit 180 for continuous delivery of an auxiliary gas (e.g., supplemental oxygen) is schematically illustrated. In some embodiments, the breathing circuit 180 is used to administer a gas exchange test or a cardiopulmonary exercise test on a patient that requires supplement oxygen during the test. In some embodiments, the breathing circuit 180 is configured to allow the concentration of the auxiliary gas to be adjusted during a test.

The breathing circuit 180 includes a blender 19. The blender 19 includes an input line for receiving oxygen and input line for receiving compressed room air. In some embodiments, the blender 19 is connected to a tank containing concentrated oxygen (e.g., 29.3-100% O₂). In other embodiments, the blender 19 is connected to an oxygen concentrator. The blender 19 includes an adjusting mechanism for selecting the concentration of oxygen delivered to the patient. The blender 19 also includes an output port connected to a breathing bag 7. In some embodiments, the breathing bag 7 is made from a non-latex material. The breathing bag 7 is connected via the elbow connector 10 and the reducer 14 to the two-way directional valve 5, which allows the patient to breathe supplemental oxygen during inspiration. The opposite side of the valve is open to room air and is used to vent the patient's expired air. The bottom port is connected to a coupler 16, which is connected to a bacterial filter 15, which is connected to another coupler 13, which is connected to the expiration side of a patient interface 8. The patient interface 8 is described in more detail above. Some embodiments do not include all of the parts described above.

A significant percentage of COPD patients who desaturate below 88% oxygen saturation with exercise may require supplemental O₂ therapy. The breathing circuit 180 can be used to evaluate patients using gas exchange testing, including cardio pulmonary exercise testing, while the patient is on supplemental oxygen at a prescribed flow rate. This is not possible using conventional cardiopulmonary exercise testing equipment due to measurement limitations associated with elevated (over that of room air) FiO₂ and the pulsatile breathing waveforms required by breath-by-breath measurement methodologies. In some embodiments, the breathing circuit 180 is also used with PH patients with R to L shunting as well as HF patients.

Acute Assessment

The gas exchange testing system 100 measures changes induced by breathing a mixture of room air and an auxiliary gas (e.g., a vasodilator or supplemental oxygen). The measurements made by the gas exchange testing system 100 serve as a feedback mechanism for a doctor, patient, or operator of the system. The changes are evaluated by comparing the measurements made by the gas exchange testing system 100 while the patient is breathing room air to measurements made while the patient is breathing the auxiliary gas.

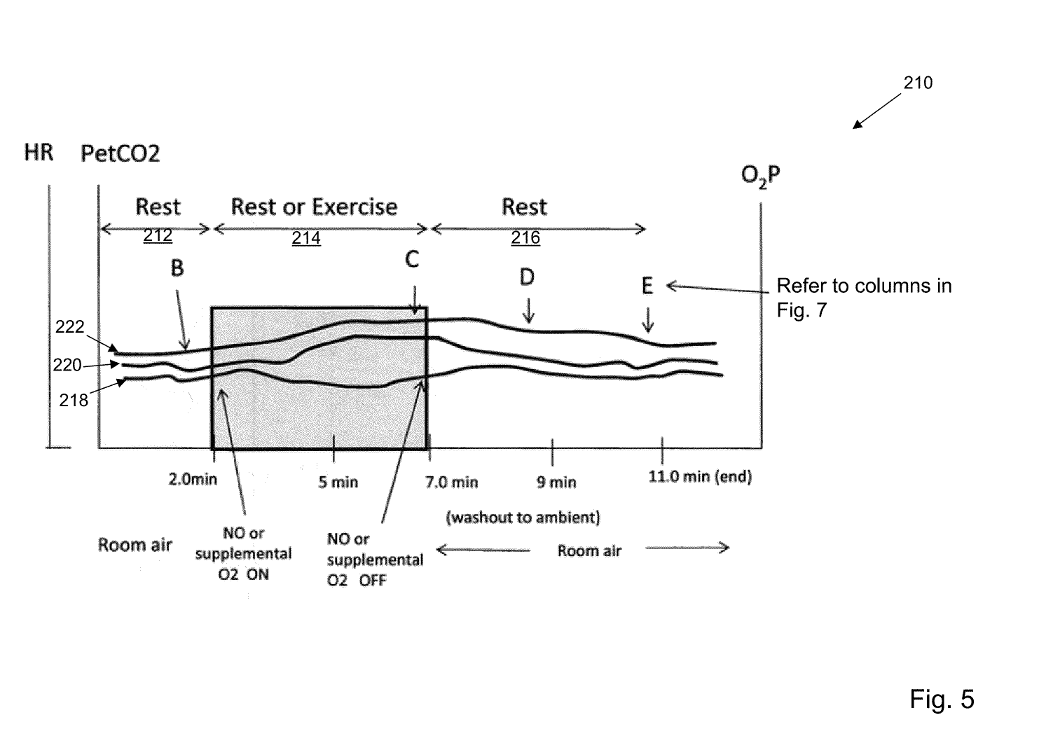

Referring now to FIG. 5, a graph 210 representing an example test protocol for evaluating the effect of breathing an auxiliary gas on a patient using the breathing circuit 142 is shown. The protocol is useful to evaluate the effect of the introduction of an auxiliary gas on the physiological parameters of a patient. In some embodiments, the auxiliary gas is a vasodilator and the protocol is used to determine if the patient is “vasoreactive” or “fixed.” A vasodilator includes any agent that causes a lowering of vascular resistance either pre- or post-capillary (e.g., by causing vasorelaxation). In other embodiments, the auxiliary gas is supplemental oxygen and the protocol is used to determine whether supplemental oxygen therapy is beneficial.

In the graph 210, time runs along the X-axis and various physiological parameters are plotted in the Y-axis. The protocol includes three phases: a pre-gas phase 212, an auxiliary gas phase 214, and a post-gas phase 216.

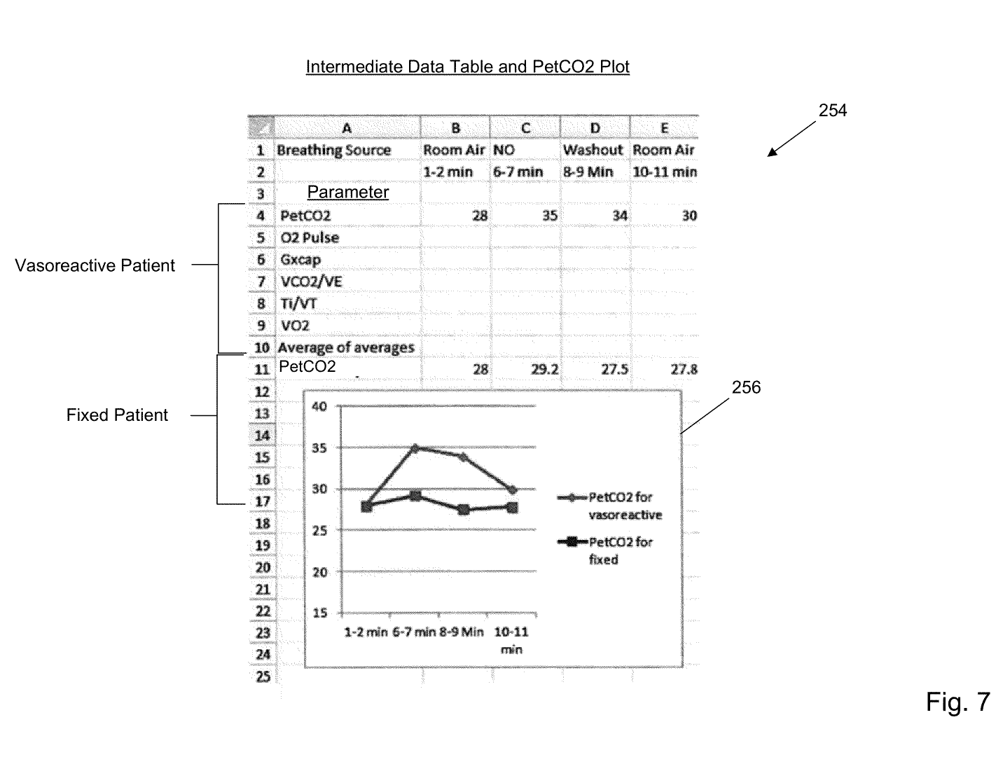

During the pre-gas phase 212, the patient breathes room air and rests. The gas exchange testing system 100 records various physiological parameters of the patient. Measurements collected during a time period near the end of the pre-gas phase (identified as B and discussed in greater detail with respect to FIG. 7) are recorded and averaged. Although in the example protocol shown in FIG. 5 the pre-gas phase lasts for two minutes, in other embodiments, the pre-gas phase lasts for a shorter or longer time.

Next, during the auxiliary gas phase 214, the patient breathes the auxiliary gas. This is accomplished by actuating the switching valve indicator switch of the stopcock 6. In some embodiments, the patient continues to rest. In other embodiments, the patient engages in light exercise. The gas exchange testing system 100 records various physiological parameters of the patient. Measurements collected during a time period near the end of the auxiliary gas phase (identified as C and discussed in greater detail with respect to FIG. 7) are recorded and averaged. Although in the example protocol shown in FIG. 5 the auxiliary gas phase lasts for five minutes, in other embodiments, the auxiliary gas phase lasts for a shorter or longer time.

Next, during the post-gas phase 216, the patient again breathes room air. In some embodiments, this is done to see how rapidly physiological parameters return to the pre-gas measurements. This is accomplished by actuating the switching valve indicator switch of the stopcock 6. Additionally, the patient rests during this phase. The gas exchange testing system 100 records various physiological parameters of the patient. Measurements collected during a time period near the end of the post-gas phase (identified as E and discussed in greater detail with respect to FIG. 7) are recorded and averaged. Additionally, measurements collected during a time period near the middle of the post-gas phase (identified as D and discussed in greater detail with respect to FIG. 7) are also recorded and averaged in some embodiments. Although in the example protocol shown in FIG. 5 the post-gas phase lasts for four minutes, in other embodiments, the post gas phase lasts for a shorter or longer time.

Using the valve indicator switch of the stopcock 6, the breathing source for the patient during a vasoreactivity test can be selected from room air and a NO/room air mixture. When room air is selected, the source of room air is through the selectable port of the three-way stopcock 6 that is open to room air, then through the breathing tube 12, through the inspiratory side of the two-way valve 5, then through the couplers 13, 16, bacterial filter 15, and patient interface 8 to the patient, who has been fitted with the mouthpiece. When the patient expires, the expired breath is directed to the expiratory side of the two-way valve 5.

When the NO/room air mixture is selected, the source of the patient's inspired air is the content of the breathing bag 7 and is delivered as described above for room air. In this manner, the source of inspiration can be selectively chosen, and the deadspace of the expiration circuit is limited to the circuit sections between the two-way valve 5 and the mouthpiece of the patient interface 8. In this manner, the undesirable effects associated with rebreathing expired air are eliminated, while the desirable effect of flexible positioning for connection of the circuit 142 to the patient interface 8 is maintained.

The graph 210 also includes plots of some exemplary measured variables, HR 218, PetCO₂ 220, and O₂ Pulse 222, for a vasoreactive patient. In some embodiments, this graph is displayed on the display terminal 114 during the test. Further, in some embodiments, the graph 210 is included in a report that summarizes the test. In some embodiments, the measurements of some or all of the physiological parameters are smoothed (e.g., by using a mid-5-of-7 filter) before being displayed on a graph used in subsequent computations.

The graph 210 is exemplary of the physiological parameters of a patient who is vasoreactive. For a patient whose response to the vasodilator is fixed, there will be very little change to these measured variables when the patient is switched from breathing room air to breathing the NO/room air mixture. In some embodiments, the operator of the gas exchange testing system 100 toggles between various user interface screens to view these results and other information about the test.

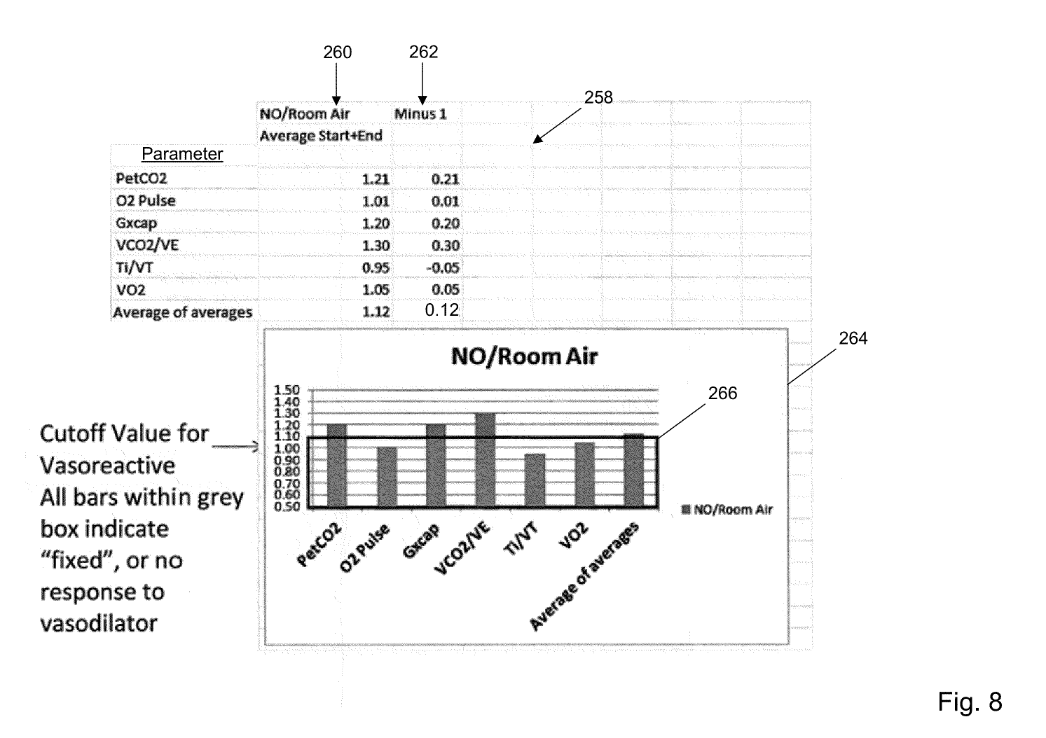

FIG. 7 shows an example data table 254 and graph 256 that embodiments of the gas exchange testing system 100 create. In some embodiments, the data table 254 is used to store results of a test performed according to the protocol illustrated by the graph 210. The rows of the data table 254 represent parameters that are measured or calculated during a test. In the example shown, the parameters stored in the data table 254 are PetCO₂, O₂ Pulse, Gx_{CAP}, VCO₂/VE, Ti/VT, and VO₂. In some embodiments, the parameters are stored in the table such that higher values are generally correlated to positive physiological findings. For example, VCO₂/VE and Ti/VT rather than VE/VCO₂ and VT/Ti are stored in data table 254 so that a measurement associated with a better physiological outcome results in a higher value for these parameters. This may be useful for graphing, averaging values, and comparing values to a threshold. Additionally, a row stores the average of the averages. The calculation of this value is explained in greater detail with respect to FIG. 8. In some embodiments, fewer, different, or additional parameters are stored in data table 254 as well.

The columns in the example data table 254 represent one-minute average values for each of the parameters for the time periods indicated in columns B, C, D, and E. However, in some embodiments, the values in the data table 254 represent averages over longer or shorter intervals. Additionally, in some embodiments, the data table 254 includes cells for additional time periods. Further some embodiments of data table 254 do not store values from all of time periods B, C, D, and E.

In the example data table 254 shown, rows 4-10 represent values from a vasoreactive patient, while later rows represent a fixed patient. In the example shown, only the first row (row 11, representing PetCO₂) of the fixed patient is shown. Similarly, data is only shown in the first row (row 4, representing PetCO₂) of the vasoreactive patient. As can be seen by the exemplary data, the vasoreactive patient's measurements change in response to the change in inspired gas, while the fixed patient's measurements do not. In this manner, the test results can be considered to determine whether a patient is vasoreactive or fixed.

Generally, values are measured or computed for all cells of the table. In some embodiments, a graph of some or all of the parameters is provided to the physician as a part of the test report summary. For example, graph 256 plots the values for PetCO₂ measured during a test of a fixed patient and a vasoreactive patient.

FIG. 8 shows an example of another data table 258 that some embodiments of the gas exchange testing system 100 create. Also shown is a bar graph 264 of the data. For each of the parameters listed in FIG. 7, three ratios are computed:
1. The average value of the parameter measured at C divided by the average value of the parameter measured at B;
2. The average value of the parameter measured at C divided by the average value of the parameter measured at E; and
3. The average value of the parameter measured at C divided by the average value of the parameter measured at B and E.

As illustrated in figured 8, ratio number 3 above is stored in the data table 258 in a first column 260. In some embodiments, the other ratios are stored as well. The second column 262 stores these ratios after they have been reduced by a value equal to the effective cutoff point (in the example shown, the effective cutoff point is 1.0). The positive values in the second column 262 are indicative of a vasoreactive patient (i.e., a ratio that is greater than the effective cutoff point). The negative values are indicative of a fixed patient (i.e., a ratio that is less than the effective cutoff point). The last row is the average of the averages. In some embodiments, this average of the averages is included on a report summary, which a physician might consider as an index of vasoreactivity. In some embodiments, a bar graph 264 representing the computed ratios in relation to a zone 266 of the chart corresponding to the fixed response is also included. Bars that extend above this zone, the top of which is the cutoff point for vasoreactivity, show which variables can easily be identified as vasoreactive indicators.

Although FIGS. 7 and 8 are discussed with respect to determining whether a patient is vasoreactive or fixed by comparing physiological parameters measured while a patient is a breathing a vasodilator to those measured while a patient is breathing room air, the same methodology is used with other auxiliary gasses in other embodiments as well. For example, in some embodiments, the method is used to determine whether providing supplemental oxygen provides a therapeutic benefit to a patient by comparing physiological parameters measured while a patient is breathing supplemental oxygen to those measured while a patient is breathing room air.

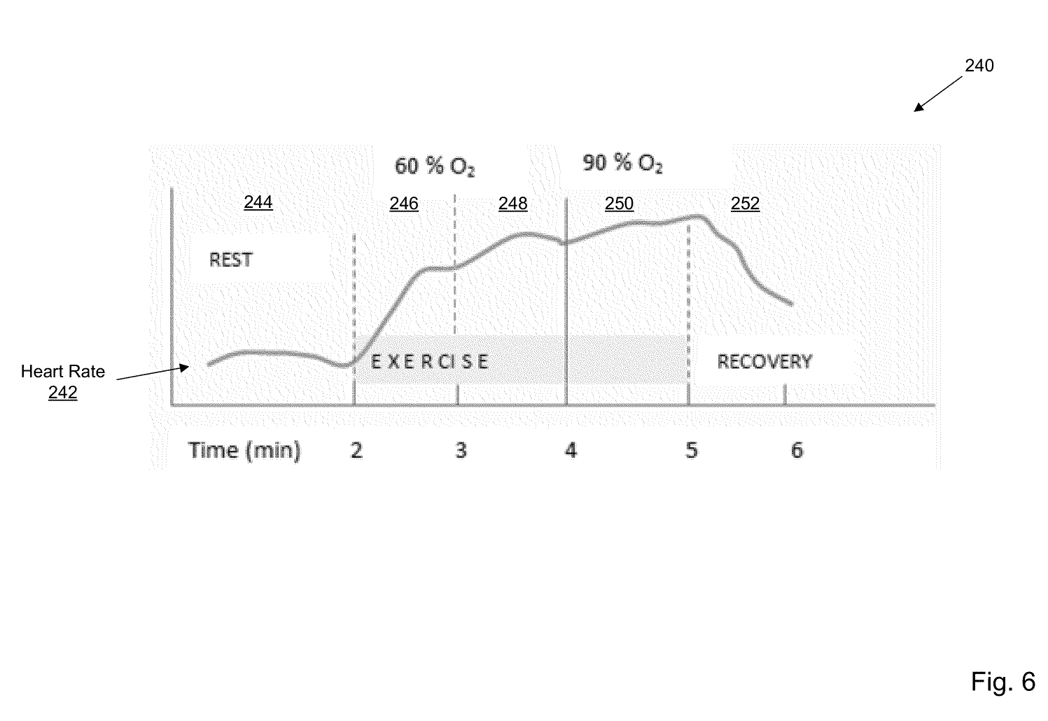

Referring now to FIG. 6, a graph 240 representing an example cardiopulmonary exercise test protocol for comparing the effect of breathing different concentrations of supplemental oxygen using the breathing circuit 180 is shown. The protocol is useful to evaluate the effect of the introduction of an auxiliary gas on the physiological parameters of a patient. In some embodiments, the auxiliary gas is supplemental oxygen and the protocol is used to determine an optimal concentration of supplemental oxygen for the patient. In other embodiments, the auxiliary gas is another gas.

In the graph 240, time runs along the X-axis and heart rate 242 is plotted in the Y-axis. The example protocol shown in FIG. 6 includes four phases: rest 244, dynamic exercise 246, first steady-state exercise 248, second steady-state exercise 250, and recovery 252.

During the rest phase 244, the patient rests while the gas exchange testing system 100 measures and records various physical parameters of the patient. During the rest phase 244, the patient breathes a first concentration of supplement oxygen (e.g., 60% O₂). Although shown in FIG. 6 as lasting for two minutes, in some embodiments, the rest phase 244 is longer or shorter than that.

Next, during the dynamic exercise phase 246, the patient begins to exercise while the gas exchange testing system 100 measures and records various physical parameters of the patient. Generally, the patient exercises at a mild intensity that can be maintained for the duration of the test. During the dynamic exercise phase 246, the patient continues to breathe a first concentration of supplement oxygen. During the dynamic exercise phase, the physiological parameters of the patient are adjusting to the demands of exercise. Accordingly, the gas exchange testing system 100 measures the physiological parameters of the patient changing. Because the physiological parameters are changing due to the demands of exercise, these measurements are not useful for comparative purposes. Although shown in FIG. 6 as lasting for one minute, in some embodiments, the dynamic exercise phase 246 is longer or shorter than that.

Next, during the first steady-state exercise phase 248, the patient continues to exercise without changing intensity from the dynamic exercise phase 246. The gas exchange testing system 100 measures and records various physical parameters of the patient. Additionally, during the first steady-state exercise phase 248, the patient continues to breathe a first concentration of supplement oxygen. During the first steady-state exercise phase, the physiological parameters of the patient are typically stable. Accordingly, the values recorded by the gas exchange testing system 100 are recorded and are useful for comparative purposes. Although shown in FIG. 6 as lasting for one minute, in some embodiments, the first steady-state exercise phase 248 is longer or shorter than that.

Next, during the second steady-state exercise phase 250, the patient continues to exercise without changing intensity from the first steady-state exercise phase 248. The gas exchange testing system 100 measures and records various physical parameters of the patient. Additionally, during the second steady-state exercise phase 250, the patient breathes a second concentration of supplement oxygen (e.g., 90% O₂). The concentration of supplemental oxygen is controlled by adjusting the blender 19. During the second steady-state exercise phase, the physiological parameters of the patient are substantially stable (after brief adjustment for changing oxygen levels). Accordingly, the values recorded by the gas exchange testing system 100 are recorded and are useful for comparative purposes. In some embodiments, the measurements made during the last thirty seconds of the second steady-state exercise phase are averaged for comparative purposes. Although shown in FIG. 6 as lasting for one minute, in some embodiments, the second steady-state exercise phase 250 is longer or shorter than that.

Next, during the recovery phase 252, the patient continues to exercise without changing intensity from the first steady-state exercise phase 248. The gas exchange testing system 100 measures and records various physical parameters of the patient. In some embodiments, the patient continues to breathe the second concentration of supplement oxygen. In other embodiments, the patient returns to breathing the first concentration of supplemental oxygen. Although shown in FIG. 6 as lasting for one minute, in some embodiments, the recovery phase 252 is longer or shorter than that.

Although FIG. 6 illustrates comparing the effect of breathing two different concentrations of supplemental oxygen, some embodiments compare the effect of breathing a single concentration of supplemental oxygen to breathing room air.

At least a portion of the physiological parameters measured during the first steady-state exercise phase 248 are compared to at least a portion of the physiological parameters measured during the second steady-state exercise phase 250. In some embodiments, the values measured during the final thirty seconds of each phase are averaged and then compared.

In some embodiments, the testing protocol described above can be used to determine: 1) whether and how much supplemental oxygen improves the patient's exercise tolerance, and 2) which of multiple concentrations of supplemental oxygen is most beneficial to the patient.

Currently, the prescription of supplemental O₂ is based solely on desaturation criteria and subjective patient input. Using the protocol described above, an operator can quantitatively evaluate, using known and clinically accepted gas exchange variables, whether an increase in inspired O₂ actually improves the take up and transport of O₂ to the peripheral muscles and respiratory muscles in order to meet the body's demands. Additional the protocol can be used to address the issue of potential O₂ toxicity by allowing the titration of O₂ concentration to determine the most effective amount of O₂ for each patient, which may not be 100% O₂ for each patient.

In current clinical practice, patients are tested using cardiopulmonary exercise testing using a six minute timed protocol similar to the protocol illustrated in FIG. 6 to assess potential cause of dyspnea and evaluated the severity of their cardiopulmonary derangement. Traditionally, the test is performed with the patient breathing room air throughout. For patients with COPD who desaturate with light exercise, such a test is frequently terminated because of the desaturation and ensuing intolerance by the patient to complete the test. In this case the first determination can be considered to be binary—was the patient able to complete the test?

Using the breathing circuit 180, the patient can now be retested while breathing supplemental oxygen to 1) determine whether the patient can now complete the test with supplemental oxygen, and 2) compare patient physiology with multiple concentrations of supplemental oxygen. Multiple concentrations of supplemental oxygen can be switched into the breathing bag during the exercise phase of the test, as illustrated in FIG. 6.

To accomplish this, in some embodiments, the gas exchange testing system measures the actual FIO₂. In other embodiments, the gas exchange testing system 100 receives a value indicative of the oxygen concentration being delivered to the patient. Further, in some embodiments, the gas exchange testing system 100 is configured to adjust the concentration of oxygen delivered to the patient according to a testing protocol. In any case, the gas exchange testing system 100 uses the actual FIO₂ value (not an assumed value representative of room air) in calculating VO₂.

The gas exchange testing system 100 measures many parameters that typically change when breathing supplemental oxygen. For example, the ventilatory efficiency slope should decrease with supplemental O₂ due to less lactate production from improved skeletal muscle tissue perfusion. The oxygen uptake efficiency slope should increase due to the increased uptake of oxygen in the lung and transport by the heart to the peripheral tissue. Oxygen saturation (SaO₂) should increase due to the increased take-up of oxygen in the lungs or improved diffusion gradient for O₂ from the alveoli across to the capillaries surrounding the alveoli. Gas exchange capacitance, Gx_{CAP} should increase, due to the increase in VO₂ and attenuation in the compensatory increased heart rate when the patient breathes room air. The end tidal partial pressure of CO₂, PetCO₂, should increase due to less hypoxic stimulation and vasoconstriction of the pulmonary vasculature. The ventilation-perfusion coupling, which is typically low during rest and exercise in COPD patients, should improve. The Shape Score (or MVI) would decrease due to the improvement in the individual variables listed above which are components of the Shape score. In some embodiments, the gas exchange testing system 100 measures some or all of the above listed parameters to evaluate the effect of supplemental oxygen therapy on the parameter for the patient.

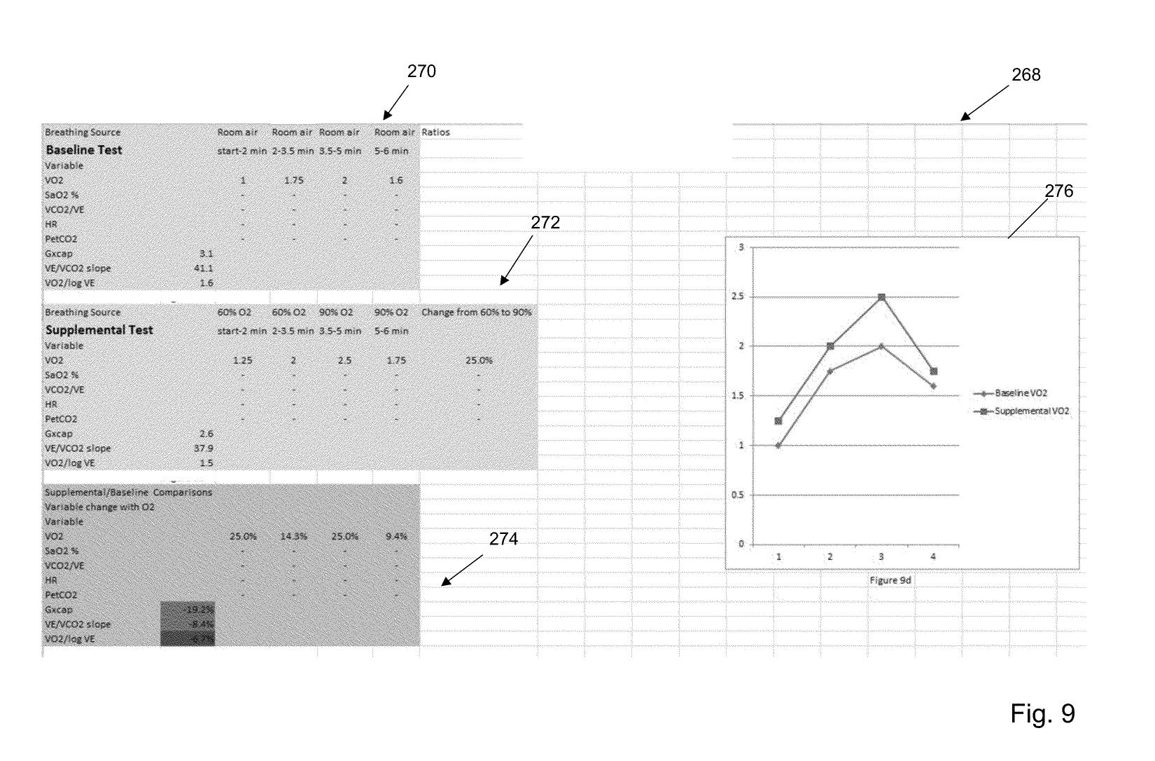

FIG. 9 illustrates an example report 268 that includes some of the data collected using the testing protocol of FIG. 6. The report includes a baseline data table 270, a supplemental O₂ data table 272, and a comparison data table 274. The baseline data table 270 contains values collected during a test without supplemental oxygen. The supplemental O₂ data table 272 contains values collected during a test where one or more concentrations of supplemental O₂ was provided. The baseline data table 270 and the supplemental O₂ data table 272 contain the average value of the parameters shown during the last thirty seconds of each phase. In the example, only VO₂ values are displayed in the report, even though cells for other variables are included in the table. Additionally, the values for the slopes and Gx_{CAP} are also computed and displayed for each test.

Supplemental O₂ data table 272 also displays the present change in each variable when supplemental O₂ is changed from 60% to 90%.

Additionally, the comparison data table 274 shows a comparison between the values collected during a baseline test (no supplemental O₂) and a test where supplemental O₂ was provided. The values for the difference between each variable are expressed as a percentage change by subtracting the baseline value from the supplemental O₂ value and dividing by the Supplemental O₂ value. If the change for GX_{CAP}, the VE/VCO₂ slope, and the VO₂/log VE slope showed an improvement in the variable, the value is highlighted in green or, if worsened, the variable is highlighted in red.

The graph 276 plots VO₂ during the baseline test and the supplemental O₂ test. Although only VO₂ is plotted in the example shown, in other embodiments other variables are plotted. In the graph 276, time runs along the X-axis and measured or computed parameters are plotted against the Y-axis. In some embodiments, the gas exchange testing system 100 also measures and plots end tidal partial pressure of O₂.

Additionally, in some embodiments, the protocol described in FIG. 6 is used to analyze other similar variables, such as the ventilation-perfusion coupling and peak VT.

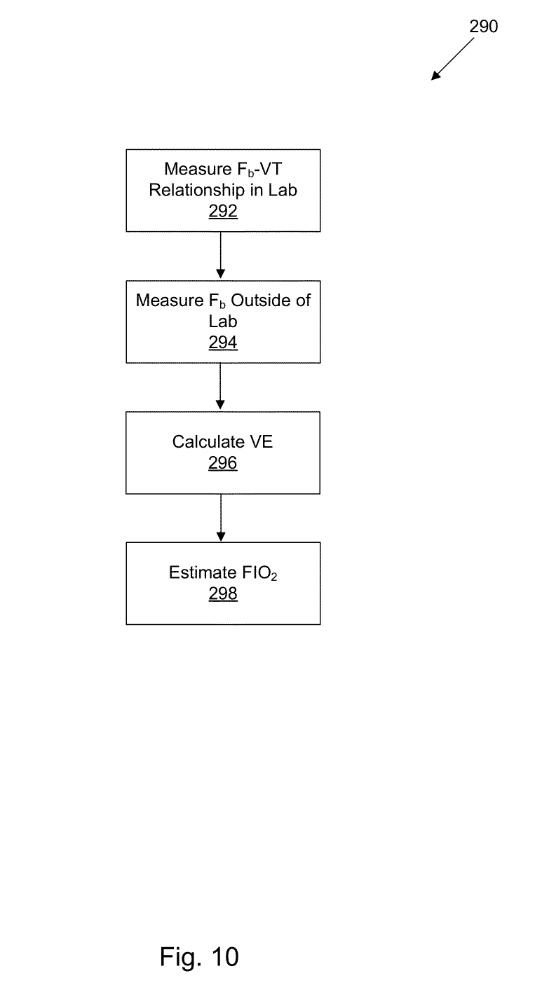

Referring now to FIG. 10, an example process 290 of estimating FiO₂ received by a patient breathing supplemental oxygen outside of a laboratory environment is illustrated.

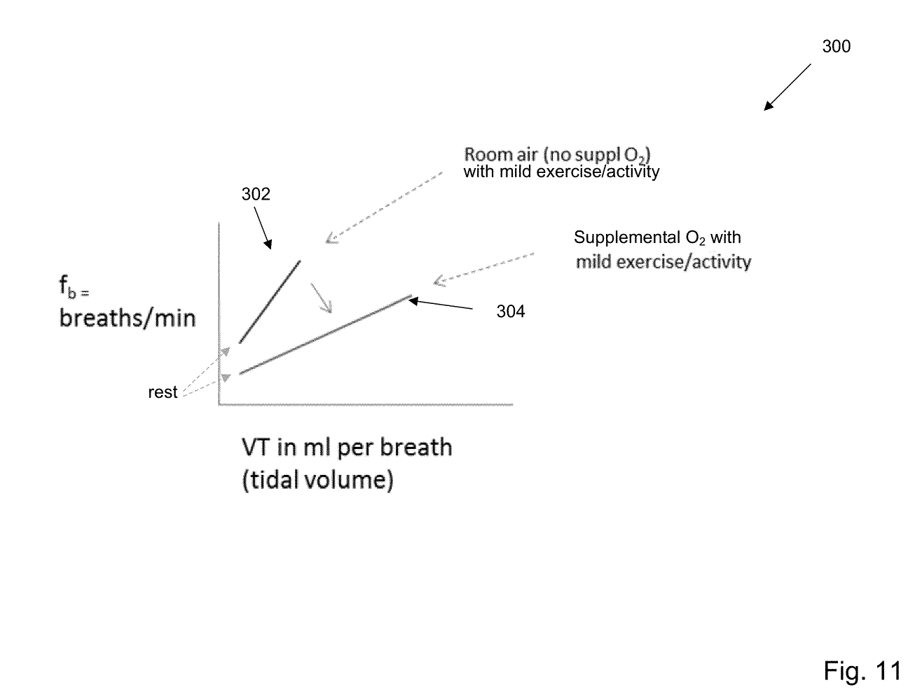

Initially, at step 292, the relationship between F_{b} and VT is measured in the laboratory using the gas exchange testing system 100 and the breathing circuit 180. The blender 19 is configured to provide the same flow volume of supplemental oxygen the patient receives outside of the laboratory. Then the F_{b} and VT of the patient are measured under a resting condition and an exercise condition while the patient receives the supplemental oxygen. A line is then fit to these two points to determine a relationship between F_{b} and VT. Examples of these lines are illustrated and discussed in greater detail with respect to FIG. 11.

Next, at step 294, the patient is evaluated outside of the laboratory setting. That is, the patient is evaluated without being connected to the gas exchange testing system 100. Instead, the patient is evaluated while using his or her typical supplement oxygen source and settings (e.g., an oxygen concentrator or oxygen tank). The F_{b} can be determined by counting the patient's breaths for one minute. In some embodiments, the oxygen concentrator detects the beginning of inspiration and automatically counts breaths to determine the F_{b}.

Next, at step 296, the minute ventilation (VE) is calculated for the patient. The minute ventilation is the volume of gas inspired during a minute. The minute ventilation is equal to the product of VT and F_{b}. F_{b}. was determined in step 294. Using the linear relationship between F_{b} and VT, VT is determined from F_{b}. Then, VE is calculated by multiplying F_{b} by VT.

Next, at step 298, FIO₂ is estimated based on VE and the settings of the supplemental oxygen source. The total volume of O2 delivered to a patient during a minute is equal to the sum of the volume of O₂ delivered by the supplemental oxygen source and the volume of O₂ in the inspired room air.

The volume of O2 delivered by the supplemental oxygen source can be determined from its settings. For example, if the supplemental oxygen source is configured to deliver 2 liters/min of 90% O₂, the patient receives 1.8 liters/min of O₂.

Similarly, the volume of O₂ received from room air in a minute is determined from the volume of room air inspired in a minute and the O₂ concentration of the room air. The volume of room air inspired in a minute by the patient is the difference between the VE value calculated in step 296 and the total volume of gas delivered by the supplemental oxygen source. For example, if the VE value calculated in step 296 is 4 liters and the supplemental oxygen source delivers 2 liters/min, the patient is inspiring 2 liters of room air per minute. The volume of O₂ is calculated based on measuring or approximating the O₂ concentration of room air. Generally, room air contains approximately 21% O₂.

Accordingly, using the calculations described above, the total volume of oxygen inspired by the patient during a minute is computed. This value is then divided by the VE value calculated in step 296 to determine the FIO₂ value.

Referring now to FIG. 11, a graph 300 is shown with two example outputs from step 292.

Line 302 illustrates the linear relationship between VT and F_{b} for an example patient breathing room air. The relationship is determined by measuring both VT and F_{b} while the patient rested and then again while the patient engaged in mild exercise/activity. Similarly, line 304 illustrated the linear relationship between VT and F_{b} for the example patient breathing supplemental oxygen. The relationship is again determined by measuring both VT and F_{b} while the patient rested and then again while the patient engaged in mild exercise/activity.

Chronic Assessment—Assessment of Patient Risk of Death

In some embodiments, upon completion of the acute phase of evaluation, the patient then enters the chronic assessment phase. The chronic assessment phase may be performed immediately after the acute assessment or at a later time or times for serial therapy tracking The chronic assessment phase evaluates the overall status of the patient's cardiac and pulmonary systems. In contrast, the acute phase evaluates the effects of breathing auxiliary gasses on the patient's cardiac and pulmonary systems.

In some embodiments, the breathing circuit 180 is used to perform chronic assessment of a patient while providing supplemental oxygen. In some embodiments, the chronic assessment is performed according to the methods discussed below.

Anderson and MacCarter have previously disclosed certain related, but different material. This includes U.S. patent application Ser. No. 12/209,376, filed Sep. 12, 2008, entitled “A Pattern Recognition System for Classifying the Functional Status of Patients with Chronic Disease”, which is hereby incorporated by reference in its entirety herein for any purpose. The application describes a method of data management for assessing a patient's autonomic balance, risk of death, and the patient's response to therapy in terms of these assessments. This method describes a process by which a set of “individual variables” are measured using the same equipment described in FIG. 1, each of which are then translated into an Individual Variable Index, and then further summed to yield a single new variable, defined as an MultiVariable Index (MVI) that quantifies the patient's disease severity. The process of selection and measurement of the MVI, and thus the sympathetic and parasympathetic components of autonomic balance during dynamic, isotonic exercise and recovery is described.

A related U.S. patent application Ser. No. 12/567,005, filed Sep. 25, 2009, entitled “A Pattern Recognition System For Classifying The Functional Status of Patients With Pulmonary Hypertension Including Pulmonary Arterial and Pulmonary Vascular Hypertension”, is also hereby incorporated by reference in its entirety herein for any purpose. That application applies a modified MVI or Multiparametric Index (MPI) which features the use of end tidal CO₂ (ETCO₂) cardiopulmonary exercise test related measurements taken over the course of an extended evaluation period to determine the presence of chronic pulmonary hypertension and classify the functional status of patients with that condition.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

What is claimed is:

1. A system for delivering an auxiliary gas to a patient during a gas exchange test, comprising:
a pneumotach;
an analyzer configured to determine a rate of a gas flow through the pneumotach; and
a directional valve having an inspiratory port and an expiratory port, the directional valve configured to connect the inspiratory port to the pneumotach during patient inspiration and to connect the expiratory port to the pneumotach during patient expiration.

2. The system of claim 1, wherein the analyzer is further configured to determine a concentration of carbon dioxide in the gas flow.

3. The system of claim 1, wherein the analyzer is further configured to determine a concentration of oxygen in the gas flow.

4. The system of claim 1, further comprising:
a breathing bag configured to provide a gas to the inspiratory port of the directional valve; and
a blender to provide a mixture of room air and the auxiliary gas to the breathing bag.

5. The system of claim 1, further comprising:
a stopcock having a first selectable port, a second selectable port, and a common port, wherein the common port is connected to the inspiratory port of the directional valve and the stopcock is configured to selectively permit flow of one of a first gas received at the first selectable port and a second gas received at the selectable port of the stopcock to the directional valve; and
a breathing bag connected to the first selectable port of the stopcock.

6. The system of claim 5, wherein the second selectable port of the stopcock is open to room air.

7. The system of claim 6, further comprising a flexible arm configured to support and position the pneumotach.

8. A method of performing a gas-exchange test on a patient, comprising:
providing an auxiliary gas to the patient; and
evaluating, using a device, a plurality of breaths of the patient while the patient is receiving the auxiliary gas,
wherein evaluating a breath of the plurality of breaths comprises:
determining a flow of gas expired by the patient during the breath; and
determining at least a portion of a composition of the gas expired by the patient during the breath.

9. The method of claim 8, wherein evaluating the breath further comprises determining at least a portion of a composition of the gas inspired by the patient during the breath.

10. The method of claim 8, wherein the auxiliary gas is supplemental oxygen.

11. The method of claim 10, wherein the plurality of breaths includes a plurality of breaths while the patient is resting and a plurality of breaths while the patient is exercising.

12. The method of claim 11, wherein determining at least the portion of the composition of the gas expired by the patient during the breath includes determining a carbon dioxide concentration in the gas expired by the patient.

13. The method of claim 12, further comprising:
calculating a ventilatory efficiency slope value for the patient.

14. The method of claim 11, wherein determining at least the portion of the composition of the gas expired by the patient during the breath includes determining an oxygen concentration in the gas expired by the patient.

15. The method of claim 14, further comprising:
calculating an oxygen uptake efficiency slope value for the patient.

16. The method of claim 11, further comprising:
calculating an average end-tidal partial pressure of carbon dioxide, an average oxygen pulse, an average ventilatory equivalent for carbon dioxide, an average inspiratory drive, an average gas exchange capacitance, and an average heart rate for the patient during the plurality of breaths while the patient is resting; and
calculating an average end-tidal partial pressure of carbon dioxide, an average oxygen pulse, an average ventilatory equivalent for carbon dioxide, an average inspiratory drive, an average gas exchange capacitance, and an average heart rate for the patient during the plurality of breaths while the patient is exercising.

17. The method of claim 8, further comprising:
providing a second gas to the patient;
evaluating a plurality of breaths of a patient while the patient is receiving the second gas; and
comparing breaths evaluated while the patient is receiving the auxiliary gas with breaths evaluated while the patient is receiving the second gas.

18. The method of claim 17, wherein the second gas is room air.

19. The method of claim 17, wherein the second gas is supplemental oxygen and an oxygen concentration of the second gas is greater than an oxygen concentration of the auxiliary gas.

20. A method of performing a gas-exchange test on a patient, comprising:
providing supplemental oxygen to the patient;
evaluating, using a device, a plurality of breaths from a rest phase and an exercise phase, the rest phase comprising a plurality of breaths while the patient is resting, the exercise phase comprising a plurality of breaths while the patient is exercising, wherein evaluating a breath comprises:
determining a concentration of oxygen in the gas inspired by the patient during the breath;
determining a flow of gas expired by the patient during the breath;
determining a concentration of oxygen in the gas expired by the patient during the breath; and
determining a concentration of carbon dioxide in the gas expired by the patient during the breath; and
calculating at least one of a ventilatory efficiency slope value and an oxygen uptake efficiency slope value for the patient based on the breaths evaluated during the rest phase and the breaths evaluated during the exercise phase.
